(19) United States
(12) Patent Application Publication
(10) Pub. No.: US 2007/0016137 A1
(43) Pub. Date: Jan. 18, 2007

(54) Infusion cannula system

(76) Inventor: Juerg Attinger, Stein am Rhein (CH)

Correspondence Address: ALCON, IP LEGAL, TB4-8, 6201 SOUTH FREEWAY, FORT WORTH, TX, 76134, US

(21) Appl. No.: 11/451,158

(22) Filed: Jun. 12, 2006

(30) Foreign Application Priority Data
Jun. 13, 2005 (EP) EP05105169.6

(51) Int. Cl. A61M 5/178

(52) U.S. Cl. 604/164.06

(57) ABSTRACT

An infusion cannula preinstalled on a surgical lance. The infusion cannula includes a “Y” or “T” fitting to which an infusion line is connected. Such a combination eliminates the need for separate handling and installation of the infusion cannula and line, and provides for a simplified insertion of the infusion cannula into an eye.

### BACKGROUND OF THE INVENTION

This invention relates generally to the field of microsurgery and, more particularly, to ophthalmic microsurgery.

During ophthalmic microsurgery, it is often necessary to remove, dissect, cut, delaminate, coagulate or otherwise manipulate delicate tissues within the eye. Microsurgical tools, such as vitreous cutters, infusion cannulas, micro scissors, micro forceps, illuminated laser probes and other devices are generally used. These devices generally are inserted through one or more surgical incisions in the sclera, a sclerotomy, and different tools may be used during different parts of the surgical procedure. The repeated insertion and removal of these instruments can allow vitreous humor and fluids to escape the eye out through the sclerotomy, increasing the potential for softening of the globe, bleeding, traction on the retina and/or introduction of bacteria into the eye as well as increased healing time. Accordingly, during ophthalmic surgery, infusion cannulas are often used to infuse an irrigating solution, such as a balanced salt solution, into the globe to maintain suitable pressure within the globe. Prior art infusion cannulas generally are inserted into the eye by first making an incision into the eye with a knife of lance. The infusion cannula is then inserted into the incision using a forceps because of the extremely small size of infusion cannula (on the order of 0.5 mm to 1.0 mm in diameter). The infusion supply line needs to be primed and then is attached, once again using a pair of forceps due to the small size cannula and the infusion line. Only then can the infusion line and infusion cannula be activated.

Therefore, a need continues to exist for a simple, inexpensive combination of infusion cannula and insertion instrument.

### BRIEF SUMMARY OF THE INVENTION

The present invention improves upon the prior art by providing an infusion cannula preinstalled on a surgical lance. The infusion cannula includes a “Y” or “T” fitting to which an infusion line is connected. Such a combination eliminates the need for separate handling and installation of the infusion cannula and line, and provides for a simplified insertion of the infusion cannula into an eye.

Accordingly, one objective of the present invention is to provide a simple, inexpensive infusion cannula.

Another objective of the present invention is to provide a simple, inexpensive infusion cannula that is easy to install.

Another objective of the present invention is to provide a simple, inexpensive infusion cannula that is preinstalled in an installation lance

Still another objective of the present invention is to provide a simple, inexpensive infusion cannula having a preinstalled infusion line.

These and other advantages and objectives of the present invention will become apparent from the detailed description and claims that follow.

### BRIEF DESCRIPTION OF THE DRAWING

FIG. 1 is a perspective view of the infusion cannula system of the present invention.

FIG. 2 is a cross-sectional view of the infusion cannula system of the present invention.

FIG. 3 is an enlarged cross-sectional view of the infusion cannula of the present invention.

FIG. 4 is an enlarged cross-sectional view of the infusion cannula of the present invention taken at circle 3 in FIG. 2 illustrating the insertion of the infusion cannula into an eye.

FIG. 4 is an enlarged cross-sectional view of the infusion cannula of the present invention illustrating the removal of the installation lance from the infusion cannula.

### DETAILED DESCRIPTION OF THE INVENTION

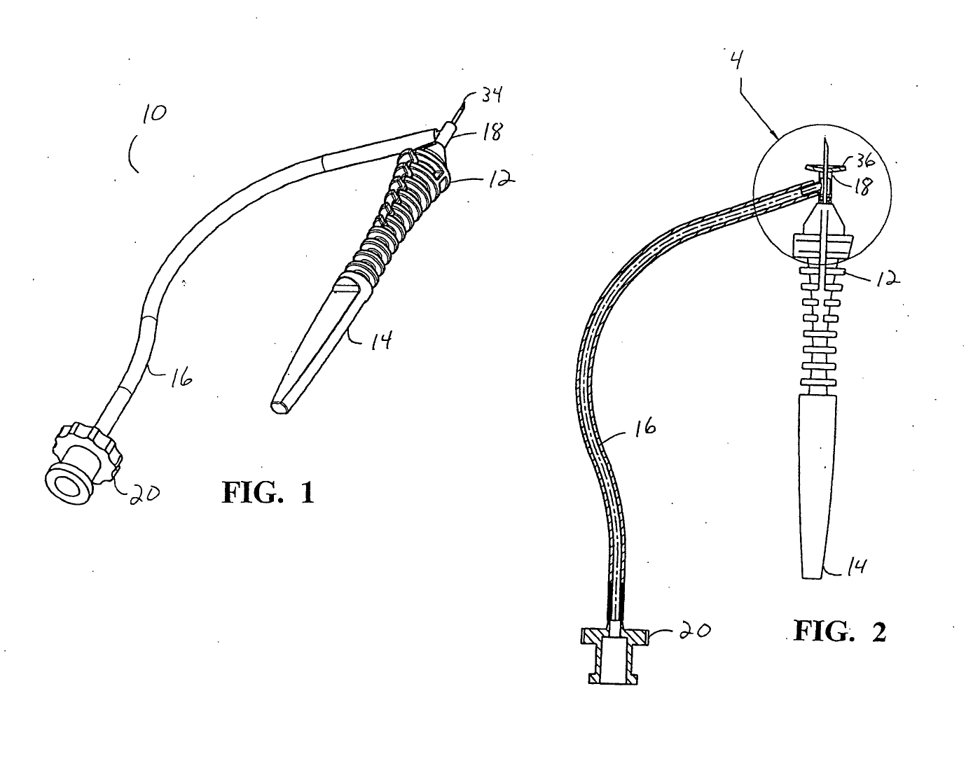

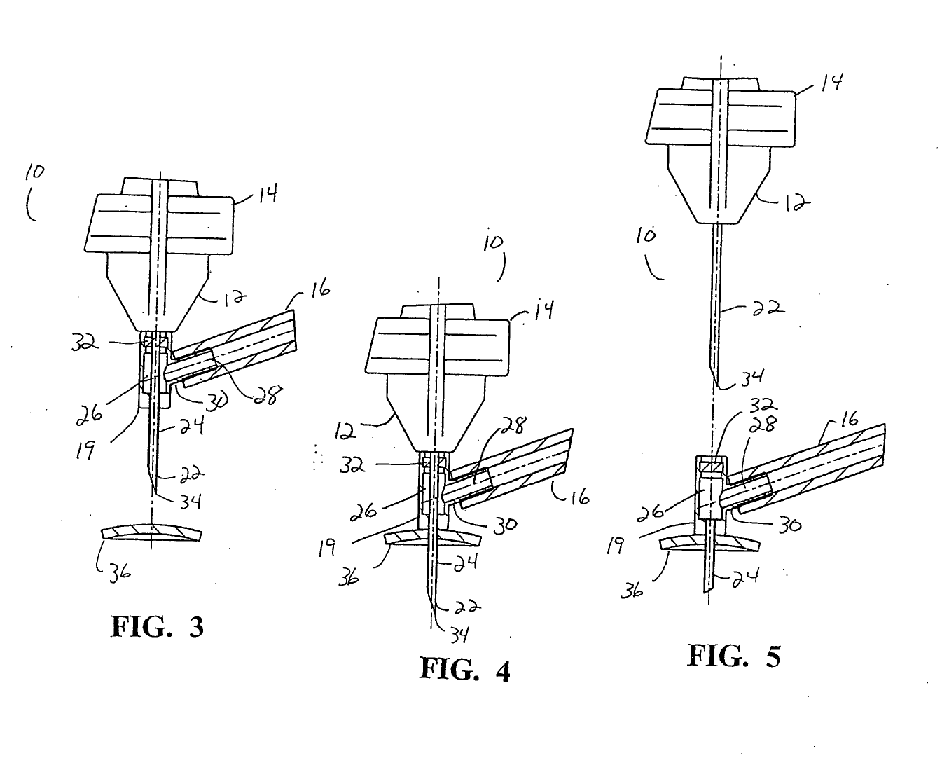

As best seen in FIGS. 1 and 2, infusion cannula system 10 of the present invention generally include lance 12 having handle 14, infusion line 16 and infusion cannula 18. Infusion line 16 is fluidly connected to infusion cannula 18 on one end, and contains a suitable connector 20, such as a luer fitting, on the opposite end for connection to a source of irrigation or infusion fluid. As best seen in FIGS. 3-5, infusion cannula 18 is generally defined by hollow, tube-like body 18 defining central, longitudinal bore 26 that fluidly communicates with tube 24 of infusion cannula 18. Infusion cannula 18 further contains side bore 28, which intersects and communicates with bore 26. Side bore 28 is defined by fitting 30, which is sized and shaped to receive infusion line 16. Lance 12 contains blade 22 sized to tightly fit within tube 24 of infusion cannula 18 so as to seal tube 24 when blade 22 is installed within infusion cannula 18 yet still be removable. Blade 22 is inserted into bore 26 and tube 24 of infusion cannula 18 through puncturable seal 32 that seal bore 26 when blade 22 is removed from infusion cannula 18. Preferably, blade 22 and tube 24 are on the order of 0.5 mm to 1.0 mm in diameter.

In use, infusion cannula assembly 10 comes preassembled with blade 22 of lance 12 inserted through bore 26 and tube 24 of infusion cannula 18 so that sharp tip 34 of blade 22 projects out of tube 24 and with infusion line 16 installed on fitting 30. Tip 34 is pressed against eye 36 so that tip 34 punctures eye 36 and blade 22 and tube 24 enters eye 36, as seen in FIG. 4. Blade 22 of lance 18 is then removed from infusion cannula 18, as shown in FIG. 5, thereby unsealing tube 24 placing bore 26 and bore 28 in fluid communication with the interior of eye 36 through tube 24. Infusion fluid from infusion line 16 can now be introduced into eye 36 with no further manipulations of infusion cannula 18 and without the need to make further connections to infusion cannula 18. Infusion fluid is prevented from exiting out of infusion cannula 18 by seal 32.

This description is given for purposes of illustration and explanation. It will be apparent to those skilled in the relevant art that changes and modifications may be made to the invention described above without departing from its scope or spirit.

I claim:

1. An infusion cannula system, comprising:
a) an infusion cannula having a hollow body defining a central bore connected on one end to a tube, the body further containing a fitting defining a side bore intersecting the central bore, the fitting in fluid communication with the tube;
b) a lance having a blade, the blade sized to fit into and be removable retained by the central bore and the tube of the infusion cannula; and
c) an infusion line connected to the fitting.

2. The infusion cannula system of claim 1 wherein the tube is located on one side of the central bore and the other side of the central bore is closed by a puncturable seal.

3. The infusion cannula system of claim 2 wherein the lance is inserted into the tube through the central bore and the seal so as to project out of the tube.

4. An infusion cannula system, comprising:
a) an infusion cannula having a hollow body defining a central bore connected to a tube on one end and closed by a seal on the opposite end, the body further having a fitting defining a side bore intersecting the central bore, the fitting in fluid communication with the tube;
b) a lance having a blade, the blade removably installed through the seal, the central bore and the tube; and
c) an infusion line connected to the fitting.
